(19) United States
(12) Patent Application Publication
(10) Pub. No.: US 2024/0099735 A1
(43) Pub. Date: Mar. 28, 2024

(54) SURGICAL INSTRUMENT

(71) Applicant: G. Hipp & Sohn GmbH, Neuhausen ob Eck (DE)

(72) Inventors: Kai WISSNER, Emmingen-Liptingen (DE); Jan BISSERT, Orsingen-Nenzingen (DE)

(73) Assignee: G. Hipp & Sohn GmbH, Neuhausen ob Eck (DE)

(21) Appl. No.: 17/769,078

(22) PCT Filed: Oct. 15, 2020
(86) PCT No.: PCT/EP2020/078997

§ 371 Date: Apr. 14, 2022

(30) Foreign Application Priority Data
Oct. 15, 2019 (LU) 101444

(51) Int. Cl. A61B 17/28

(52) U.S. Cl. CPC A61B 17/2804; A61B 2017/00845

(57) ABSTRACT

A surgical instrument (1) comprises at least three individual components (2,3,4,5,6) arranged movably relative to one another. Each of the individual components (2,3,4,5,6) is directly and movably connected to at least one further one of the individual components (2,3,4,5,6). All of the constituent parts of the surgical instrument (1) are made of a metallic or metal-containing material. Each individual component (2,3,4,5,6) has at least one friction surface region (9,10) which forms a pair of friction surfaces with at least one friction surface region (9,10) of another individual component (2,3,4,5,6). At least one of the two friction surface regions (9,10) of each pair of friction surfaces has a hard-material surface layer (16).

### TECHNICAL FIELD

The disclosure relates to a surgical instrument.

### BACKGROUND

Surgical instruments are used during operations. They can be, for example, forceps, clamps or retractors, wherein the individual components can be keys, joints, screw connections, shafts, pins, springs, ratchets or sockets.

In the case of surgical instruments made of metallic or metal-containing materials, friction in the friction surface regions leads to wear in these regions. This reduces the service life of the surgical instruments, i.e. the period over which the surgical instruments can be used.

In order to reduce the friction between two friction surface regions, it is customary to use lubricants for surgical instruments and in particular for those in which the friction surface regions resting against each other are made of metal. In the case of reusable surgical instruments made of metallic or metal-containing materials, lubrication is usually performed after cleaning and disinfection and prior to sterilization and commissioning. A lubricant film of paraffin-containing lubricants is usually applied to lubricate the friction surface regions.

A particular disadvantage of using lubricants in reusable surgical instruments is that the sterility of the surface areas under the lubricant film is impaired. In addition, another disadvantage of using lubricants is that they can be washed out, particularly if the surgical instrument in question is used over a longer period of time during a prolonged operation. As a result, the lubricating effect is steadily reduced during use, while the friction between the friction surface regions increases. This can lead to sluggish behavior of the surgical instrument. Continued use of the then inadequately lubricated surgical instrument usually leads to irreversible wear and thus to failure of the surgical instrument.

Part of a reprocessing process for reusable surgical instruments usually involves heating the instruments, for example using an autoclave, to clean and sterilize them. Before the lubricating film can be applied, the surgical instruments treated in this way must cool down. This slows down the reprocessing time of the surgical instruments, which leads directly to higher reprocessing costs. A further disadvantage is that more user errors can occur, especially when surgical instruments with numerous individual components and multiple friction surface regions are the subject of the reprocessing process.

In addition, the use of lubricants has proven to be disadvantageous in that some properties of the lubricants change over time, especially under changing environmental conditions and incorrect storage, for example by becoming resinous. Such resinified lubricants can then only be removed from the surgical instrument with great effort. This increases both the time and the cost of the reprocessing of the surgical instruments.

It is known from the prior art that a hard-material surface layer can be applied to the friction surface regions of surgical instruments of the type described above in order to increase the service life of the surgical instruments compared to uncoated surgical instruments. The hard-material surface layers are applied to both friction surface regions of the pair of friction surfaces and are each made of the same material. Due to the same hardness values of the hard material particles, friction of the friction surface regions against each other leads in particular to abrasive wear of the friction surface regions and thus to a reduction in the service life of the surgical instrument. The use of a lubricant can in turn reduce abrasive wear, but this leads to the disadvantages mentioned above in the reprocessing of the reusable surgical instruments.

### SUMMARY

A surgical instrument has at least three individual components arranged movably relative to one another. Each of the individual components is directly and movably connected to at least one further one of the individual components. Each individual component comprises at least one friction surface region which forms a pair of friction surfaces with at least one friction surface region of a further individual component. The individual components are manufactured from a metallic or metal-containing material at least in portions which are associated with the friction surface regions.

An object of the disclosure is to increase the service life of the surgical instrument while at the same time reducing the amount of lubricant required.

This object is achieved in that only one of the two friction surface regions of each pair of friction surfaces comprises a hard-material surface layer which is materially-bonded to the individual component in the respective friction surface region. The hard-material surface layer has a higher hardness than the metallic or metal-containing material from which the individual components are made. The hard-material surface layer is applied in the respective friction surface region by means of suitable processes. The friction in the friction surface regions of the pair of friction surfaces is reduced in that it is provided, that a hard-material surface layer is applied in only one of the two friction surface regions of each pair of friction surfaces. In this way, the wear of the surgical instrument is reduced and its service life is increased.

Advantageously, it is provided that at least the individual components having a friction surface region are made entirely of the metallic or metal-containing material. For example, the movable components of a surgical clamp are made of titanium, wherein the clamp can have grip parts made of plastic, which can be placed over grip sections of clamping elements of the clamp. However, it is also possible and provided that all individual components are made entirely of the metallic or metal-containing material.

In order to further reduce friction and thus wear in the friction surface regions of the friction surface pairing, it is also possible and provided for a surface roughness of at least one of the two friction surface regions to be reduced by means of a surface machining process, for example by grinding, polishing, lapping or the like. It is envisaged that the surface roughness of the at least one friction surface region can be reduced both before the application of the hard-material surface layer and after the application of the hard-material surface layer. In both cases, the service life of the surgical instruments is increased by the reduced friction and the resulting reduced wear.

An advantageous implementation provides that the entire surgical instrument, except for one of the friction surface regions of each pair of friction surfaces, has a hard-material surface layer. As a result of the fact that only one of the friction surface regions of each pair of friction surfaces always has the hard-material surface layer, the friction and thus the wear in the friction surface regions of the pair of friction surfaces is reduced and the service life of the surgical instruments is increased.

It is provided that the production of the hard-material surface layer in this embodiment of the surgical instrument can be carried out, for example, by first applying the hard-material surface layer onto the entire surgical instrument, wherein in each case one of the friction surface regions of the friction surface pairing is treated by means of an ablative manufacturing process, for example by means of grinding, polishing, electropolishing or the like, and thus the hard-material surface layer is removed at least in portions in this friction surface region. In this way, the hard-material surface layer can be applied particularly easily.

In an advantageous embodiment, it is provided that at least one of the individual components has a hard-material surface layer that completely covers a component surface of the individual component, wherein at least one of the other individual components does not have a hard-material surface layer. Such a surgical instrument is particularly easy to manufacture. In particular, the manufacturing of this embodiment of the surgical instrument eliminates the need for mechanical treatment of at least one of the friction surface regions of a friction surface pairing by grinding, polishing, or the like.

In an advantageous embodiment of the surgical instrument, it is provided that one of the friction surface regions of at least one of the pairs of friction surfaces has a hard-material surface layer, the other friction surface region of the pair of friction surfaces having a soft-material surface layer the hardness of which is lower than that of the hard-material surface layer. It is thus provided that both friction surface regions have a coating.

The advantage of such a configuration of the surgical instrument is that the combination of the different hardnesses of the two friction surface regions can reduce the friction and thus the wear in the friction surface regions of the pair of friction surfaces and thus increase the service life of the surgical instruments. It is also provided that the friction surface region with the lower hardness can have a soft surface layer applied by a chemical or physical coating process. It is provided that the soft-material surface layer has a hardness which is lower than the hardness of the hard-material surface layer, it being advantageously provided that the soft material surface layer has a higher hardness than the metallic or metal-containing material from which the individual components are made.

Particularly preferably, the applied layer consists, for example, of a material which forms a polishing medium with lubricating properties due to abrasive wear. Such a material smears out of the hard-material surface layer due to friction with the friction surface region, the hard-material surface layer being smoothed as a result. By smoothing at least one of the friction surface regions of a pair of friction surfaces, the friction and thus the wear in the friction surface regions of the pair of friction surfaces is reduced and the service life of the surgical instruments is increased.

A major influencing factor in a friction process is a surface roughness of surfaces involved in the friction process. One way of indicating the surface roughness of a surface is the averaged roughness depth Rz, which is given in μm. In the surgical instrument, it is advantageously provided that the hard-material surface layer has a lower averaged roughness depth than the soft-material surface layer or an untreated surface. If a soft-material surface layer is used, it advantageously has a lower averaged roughness depth than the average roughness depth of the untreated surface of the individual component. It is advantageously provided that the soft surface layer or already the untreated surface has an average peak-to-valley height of less than 10 μm, preferably less than 6 μm and particularly preferably less than 4 μm.

An advantageous implementation provides that at least one of the hard-material surface layers is produced at least in portions by a chemical coating process. The hard-material surface layers are particularly easy to produce by means of chemical coating processes, in particular by means of plasma-assisted chemical vapor deposition (PACVD). A particular advantage of a hard-material surface layer applied by means of PACVD is that, in particular, a degree of reflection, a tendency to rust formation and the roughness of a surface area coated in this way can be reduced.

In this context, it is advantageously provided that selective coating can be achieved by means of a stencil suitable for use in the chemical coating process. The stencil can in particular be made of a metal or a plastic.

An advantageous embodiment provides that at least one of the hard-material surface layers is produced at least in portions by a physical coating process. The hard-material surface layers are particularly easy to produce by means of physical coating processes, in particular by means of physical vapor deposition (PVD). A particular advantage of a hard-material surface layer applied by PVD is that the reflectance, the tendency to rust formation and the roughness of a surface area coated in this way can be reduced.

It is advantageously provided that at least one of the hard-material surface layers produced by a chemical or physical coating process may comprise at least one of the coating materials TiN, TiAlN, ALTiN, CrN, CrCN, Cr+a-C:H:W, Cr+a-C:H:W+a-C:H, Cr+CrN+a-C:H, a-C:H, t-a:C, ZrN, MoS2, Ti, or Nb(X,O).

In this context, it is advantageously provided that selective coating can be achieved by means of a stencil suitable for use in the physical coating process. The stencil can in particular be made of a metal or a plastic.

In an advantageous embodiment of the surgical instrument, it is provided that at least one of the hard-material surface layers is made at least in portions from a metallic or metal-containing material. A metallic hard-material surface layer can preferably be made from a steel alloy, wherein a surface layer of this type can be produced particularly inexpensively.

A metallic hard-material surface layer can preferably be made of titanium or a titanium-containing material, in particular titanium nitride (TiN), wherein a hard-material surface layer made of this material exhibits low wear in the friction surface regions of the pair of friction surfaces and the service life of the surgical instruments can thus be further extended. In addition, hard-material surface layers made of TiN have a gold-colored surface, which increases the recognizability of the surgical instruments and in particular the coated surface areas in a particularly advantageous manner.

An advantageous implementation provides that at least one of the hard-material surface layers is made at least in portions from a non-metallic material. In this context, it is advantageously provided in particular that the non-metallic material is or comprises a ceramic. Ceramics and ceramic materials are characterized in particular by the fact that they are particularly wear and abrasion resistant, whereby the service life of a surgical instrument designed in this way is increased.

An advantageous embodiment provides that at least one of the individual components is manufactured at least in portions from a steel alloy. The production of at least one of the individual components at least in portions from a stainless steel alloy offers the advantage of a cost-effective production of the individual components. Stainless steel alloys are easy to machine, in particular by means of mechanical manufacturing processes, and give rise to relatively low material costs. In addition, a large number of biocompatible stainless steel alloys are available on the market, which are particularly suitable for the manufacture of medical technology products.

Furthermore, it is also provided that at least one individual component can be made, at least in portions, from a titanium base material. Titanium and titanium alloys exhibit, in particular, high biocompatibility, high mechanical strength and high corrosion resistance.

In an advantageous embodiment of the surgical instrument, it is provided that at least one component surface of one of the individual components has at least one passivation layer at least in sections, wherein at least one hard-material surface layer is applied to the passivation layer at least in sections.

The application of passivation layers to the individual components of the surgical instrument, at least in sections, offers the advantage of high corrosion resistance of the passivated component surface. Advantageously, the hard-material surface layer is applied to the passivation layer at least in sections, wherein the hard-material surface layer does not cause damage on the passivation layer. Advantageously, this increases protection of the surgical instrument against corrosion.

Furthermore, it is possible for the hard-material surface layer itself to be formed as a passivation layer, at least in sections. It is thus conceivable that the passivation layer can be replaced by the hard-material surface layer, particularly in areas of the individual components that are difficult to access. This leads to cost-effective manufacture of the surgical instrument.

It is also advantageously provided that the surgical instrument can be manufactured by means of a process in which all the individual components are first coated with a hard- and/or soft-material surface layer in a coating step, in accordance with the concept described above. Advantageously, the individual components are assembled to form the surgical instrument in an assembly step following the coating step. Preferably, the surgical instrument is used “dry” in a grinding-in step following the assembly step, wherein “dry” in the present case means that a working movement which can be performed with the surgical instrument is carried out without the surgical instrument actually being used in the course of an operation, as a result of which the surgical instrument is ground-in in the region of the pairs of friction surfaces and the friction surfaces are provided with a surface finish. Furthermore, it is advantageously provided that the surgical instrument is cleaned in a cleaning step following the grinding-in step, in particular in order to remove an excess portion of an abrasion produced during grinding-in.

### BRIEF DESCRIPTION OF THE DRAWINGS

FIG. 1 shows a schematic side view of a surgical instrument in the form of a rongeur.

FIG. 2 is a schematically-illustrated perspective view of two individual components of the surgical instrument shown in FIG. 1.

### DETAILED DESCRIPTION

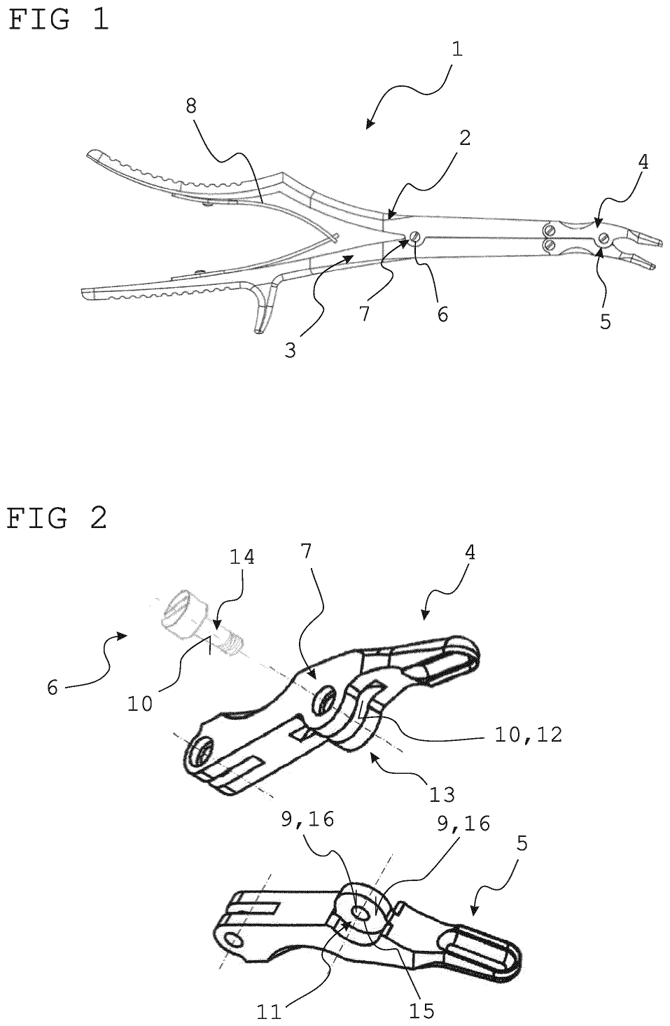

FIG. 1 shows a schematic representation of a surgical instrument 1. The surgical instrument 1 has an upper handle 2, a lower handle 3, an upper jaw part 4, a lower jaw part 5 and a connecting screw 6. These elements constitute the individual components 2,3,4,5,6 of the surgical instrument. The upper handle 2, the lower handle 3, the upper jaw part 4 and the lower jaw part 5 are directly and movably pivotally connected to each other by means of in each case one connecting screw 6 at four connecting regions 7 of the surgical instrument 1. In each of the connecting points 7, the upper handle 2 is pivotally connected to the lower handle 3, the upper handle 2 is pivotally connected to the upper jaw part 4, the lower handle 3 is pivotally connected to the lower jaw part 5, and the upper jaw part 4 is pivotally connected to the lower jaw part 5 by means of the one of the connecting screws 6.

The illustrated surgical instrument 1 is shown in an unactuated state in which the individual components 2, 3, 4, 5, 6 are transferred to an open position. The upper handle 2 and the lower handle 3 are held in the illustrated open position by a leaf spring 8. Due to the mechanical coupling of the individual components 2, 3, 4, 5, 6 in the surgical instrument, the upper jaw part 4 and the lower jaw part 5 are also held by the leaf spring 8.

When the surgical instrument 1 is actuated, by displacing the two handles 2, 3 against a spring force exerted by the leaf spring 8, the two jaw parts 5, 6 are displaced towards each other until the jaw parts 5, 6 are brought into abutment with each other.

FIG. 2 shows a schematic and enlarged exploded view of the upper and lower jaw parts 4,5 and the connecting screw 6 connecting the two jaw parts 4,5 of the surgical instrument shown in FIG. 1. The upper jaw part 4 and the lower jaw part 5 are pivotably connected to each other in the connecting region 7 by means of the connecting screw 6.

A plurality of pairs of friction surfaces (no reference character assigned) are formed in the connecting region 7, each having a first friction surface region 9 and a second friction surface region 10. When the individual components 2, 3, 4, 5, 6 are displaced relative to each other, the friction surface regions 9, 10 of the pair of friction surfaces rub against each other. First friction surface regions 9 are formed on a hinge element 11 of the lower jaw part 5. Second friction surface regions 10 are formed on opposite inner sides 12 (only one designated) of a hinge receptacle 13 of the upper jaw part 4, and on a shank portion 14 of the connecting screw 6. The hinge element 11 is disposed within the hinge receptacle 12, wherein the connecting screw 6 is passed through a screw recess 15 of the hinge element 11 so that the first friction surface 9 as well as the second friction surface 10 are brought into abutment with each other.

In the illustrated embodiment of the surgical instrument, the first friction surface regions 9 comprise a hard-material surface layer 16.

In the illustrations in FIGS. 1 and 2, individual elements of multiple similar elements are indicated with a reference character as an example.

### LIST OF REFERENCE CHARACTERS

1. surgical instrument
2. upper handle
3. lower handle
4. upper jaw part
5. lower jaw part
6. connecting screw
7. connecting region
8. leaf spring
9. first friction surface region
10. second friction surface region
11. hinge element
12. interior surfaces
13. hinge receptacle
14. shank portion
15. screw recesses
16. hard-material surface layer

1.-11. (canceled)

12. A surgical instrument (1), comprising
at least three individual components (2,3,4,5,6) arranged movably relative to one another,
wherein each of the individual components (2,3,4,5,6) is directly and movably connected to at least one further one of the individual components (2,3,4,5,6),
wherein each individual component (2,3,4,5,6) comprises at least one friction surface region (9,10) which forms a pair of friction surfaces with at least one friction surface region (9, 10) of a further individual component (2,3,4,5,6),
wherein the individual components (2,3,4,5,6) are manufactured from a metallic or metal-containing material, at least in portions which are associated with the friction surface regions (9,10),
wherein exclusively one of the two friction surface regions (9, 10) of each pair of friction surfaces comprises a hard-material surface layer (16) which is materially-bonded to the individual component (2,3,4,5,6) in the respective friction surface region (9,10).

13. The surgical instrument (1) according to claim 12,
wherein at least the individual components (2,3,4,5,6) comprising a friction surface region (9,10) are made entirely of the metallic or metal-containing material.

14. The surgical instrument (1) according to claim 12,
wherein the entire surgical instrument (1) comprises a hard-material surface layer (16) except for one of the two friction surface regions (9, 10) of each pair of friction surfaces.

15. The surgical instrument (1) according to claim 12,
wherein at least one of the individual components (2,3,4,5,6) comprises a hard-material surface layer (16) completely covering a component surface of the individual component (2,3,4,5,6), and
wherein at least one of the other individual components (2,3,4,5,6) does not comprise a hard-material surface layer (16).

16. The surgical instrument (1) according to claim 12,
wherein one of the friction surface regions (9,10) of at least one of the pair of frictions surfaces comprises a hard-material surface layer (16), and
wherein the other friction surface region (9, 10) of the pair of friction surfaces comprises a soft-material surface layer the hardness of which is lower than that of the hard-material surface layer (16).

17. The surgical instrument (1) according to claim 12,
wherein at least one of the hard-material surface layers (16) is produced at least in portions by a chemical coating process.

18. The surgical instrument (1) according to claim 12,
wherein at least one of the hard-material surface layers (16) is produced at least in portions by a physical coating process.

19. The surgical instrument (1) according to claim 12,
wherein at least one of the hard-material surface layers (16) is produced at least in portions from a metallic or metal-containing material.

20. The surgical instrument (1) according to claim 12,
wherein at least one of the hard-material surface layers (16) is produced at least in portions from a non-metallic material.

21. The surgical instrument (1) according to claim 12,
wherein at least one of the individual components (2,3,4,5,6) is produced at least in portions from a steel alloy.

22. The surgical instrument (1) according to claim 12,
wherein at least one component surface of one of the individual components (2,3,4,5,6) comprises at least one passivation layer at least in sections, and
wherein at least one hard-material surface layer (16) is applied to the passivation layer at least in portions.
